(19) United States
(12) Patent Application Publication
(10) Pub. No.: US 2005/0232996 A1
(43) Pub. Date: Oct. 20, 2005

(54) Matrix comprising a bioactive component containing phospholipid

(76) Inventors: Dirk Cremer, Zolling (DE); Elisabeth Markl, Rohrmoos (DE)

Correspondence Address: FITCH EVEN TABIN AND FLANNERY, 120 SOUTH LA SALLE STREET, SUITE 1600, CHICAGO, IL, 60603-3406, US

(21) Appl. No.: 10/511,888

(22) PCT Filed: Apr. 17, 2003
(86) PCT No.: PCT/EP03/04030

§ 371 Date: Oct. 19, 2004

(30) Foreign Application Priority Data
Apr. 19, 2002 (DE) 102 17 558.6
Oct. 31, 2002 (DE) 102 50 727.9

(51) Int. Cl. A61K 9/14

(52) U.S. Cl. 424/484

(57) ABSTRACT

The present invention concerns a matrix with a bioactive component containing phospholipid which is composed of 5 to 98% by weight phosphatidyl serine and 1 to 90% by weight phosphatidyl choline. In addition 1 to 94% by weight of a fat component of vegetable and/or animal origin, a wax component, a polyalcohol component and/or other physiologically compatible additives can be contained. In this connection a matrix is preferred which has a permanently flexible water-containing coat and which has a total diameter of between 0.3 to 20 mm. This matrix is used especially to strengthen the ability to cope with physical and mental stress and functional capacity.

The present invention concerns a matrix with a bioactive component containing phospholipid as well as the use thereof.

The substance class of phospholipids are so-called complex lipids having amphiphilic properties i.e. they are at the same time lipophilic and hydrophilic which, among others, enables them to form lipid bilayers in aqueous media.

Phospholipids (also referred to as phosphatides) are chemically considered phosphodiesters in which the phosphoric acid is esterified, on the one hand, with a sphingosine or glyceride residue and, on the other hand, with choline, ethanolamine, serine, inositol or glycerol. Phosphatidyl choline is also known as lecithin and at the same time is an eponym for a large group of special phospholipids, the lecithins. Phosphatidyl serine and phosphatidyl ethanolamine are also referred to as cephalins.

The lyso derivatives which also belong to this group are formed by hydrolytic cleavage by means of specific phospholipases.

Phospholipid-containing capsules are sufficient well-known from the prior art and contain phospholipids mostly as a coating substance. If phospholipids are used in the filling, i.e. in the core of the capsule, they mostly act there in small amounts as a formulation adjuvant usually having solubilizing properties.

As a result of their amphiphilic properties phospholipids are also used as coating substances of the known liposomes and transferosomes. In this connection they are used especially in the field of mucosal applications due to their bioadhesive properties where they are introduced especially into nasal and oral cavities.

However, in a chemically-modified form phospholipids are also used as surface-active formulation adjuvants (surfactants).

It is also known that vesicles which carry phospholipids as a coat can be produced by means of ultrasound.

Special granulates with lecithin coats are known from the Japanese application JP 91 47 043 as well as from EP-A 493 441. These granulates which, among others, contain steroids as bioactive substances are used as feed additives.

According to WO 87/04347 lyso-phospholipids are described as solubilizers for hydrophobic bioactive substances.

Pharmaceutical forms that can enter the lungs which use organic halogen compounds as a carrier for the phosphatidyl choline are described in the International Applications WO 99/16419 and 99/16421.

Soft gelatin capsules which are commercially available as KAL®-Lecithin and which contain 1200 mg soybean lecithin contain lecithin as a bioactive ingredient. However, in order to accommodate this amount of lecithin in a capsule, capsule sizes have to be selected which come close to the centimetre limit and thus give rise to a limited compliance.

A process for producing phosphatidyl serine (PS), i.e. a phospholipid, is known from the German Patent DE 199 17 249. In this connection it is stated that the PS or corresponding PS products obtained in this manner can be stabilized in aqueous systems by embedding them in a hard fat. However, the proposals made there are limited to soft gelatine capsules which should have the special PS in their contents. Although the described system stabilizes phosphatidyl serine which is known to be hydrolytically unstable, it has the disadvantage that this formulation cannot be encapsulated.

Formulations of phosphatidyl serine and phosphatidyl choline especially in a mixture with other lecithins and/or oils have proven to be not sufficiently stable in soft capsules. Also there are very tight constraints on the mixing (blending) with regard to the melting point and flow behaviour of the capsule contents due to the technical requirements of the encapsulation which is effected typically by means of a so-called rotary dye. Also the processing temperatures in the encapsulation process often have an adverse effect on the properties of the capsule coat and/or the capsule contents.

One result of this described instability of the lecithins as capsule contents is that at best moderately pasty blends can be encapsulated instead of a desired liquid formulation.

In addition the encapsulation of phospholipids in general causes major problems since, as described, they also act as emulsifiers and thus rapidly effect in a mixing of the not yet hardened (dried) coat with the contents during the encapsulation process. As a result the capsules become permeable within a relatively short time, they leak and can thus no longer be used.

Hence the object of the present invention was to provide a matrix with a bioactive component containing phospholipid which does not have the disadvantages of the described prior art and which can be formulated in an economically acceptable manner. Moreover, the bioactive phospholipid component introduced into the matrix should have an adequate stability for the most common purposes of application even in an encapsulated state.

This object was achieved by an appropriate matrix which contains as a bioactive component 5 to 98% by weight phosphatidyl serine (PS) and 1 to 90% by weight phosphatidyl choline (PC) and in addition 1 to 94% by weight of at least one other component from the series of fat component of vegetable and/or animal origin, wax component, polyalcohol component and other physiologically compatible additives.

Surprisingly with the matrix according to the invention it has turned out that the phosphatidyl serine and/or phosphatidyl choline portions contained therein are extremely stable towards the otherwise negative hydrolysis and/or the general degradation of encapsulated lecithins. This is especially strongly pronounced in blends that are highly viscous and additionally have thixotropic properties. Previously it was only known that highly viscous blends make lecithins more stable but it was then no longer possible to encapsulate these highly viscous mixtures (cf. DE-OS 199 17 249). This is even more surprising since it is known especially that phosphatidyl serine is significantly less stable than other phospholipids.

In addition to the head group (i.e. serine and/or choline), the compounds preferably each contain a residue at positions sn-1 and/or sn-2 which is derived from a C₂-C₃₀ carboxylic acid, in particular a C₁₂-C₂₈ carboxylic acid bound to the hydroxyl groups of the glycerol. The acid residues can be linear or branched, saturated or monounsaturated or polyunsaturated. Particularly preferred residues are residues that are formed by the binding of acetic acid, butyric acid, caproic acid, caprylic acid, capric acid, lauric acid, myristic acid, arachidic acid, behenic acid, lignoceric acid, β-linolenic acid, eicosapentaenoic acid, erucic acid, nervonic acid, α- or β-eleosteric acid or parinaric acid. Residues are particularly preferred which are formed by binding of palmitic acid, stearic acid, oleic acid, linoleic acid, α-linolenic acid, arachidonic acid or docosahexaenoic acid. The acid residues bound to the OH groups of the glycerol that are still available can thereby be identical or different.

In the total matrix according to the invention phosphatidyl serine and phosphatidyl choline represent the bioactive part; the portions of the other components give the total matrix its advantageous properties.

In the context of the present invention the term “bioactive” is understood as the effect of phosphatidyl serine and phosphatidyl choline to display a biological action in the living organism which usually applies to corresponding preparations in the human or veterinary field during or after their release from the total matrix in the resorption area, on the transport path or at the site of action. In this connection this bioactive effect is of course not limited to the two said phospholipids but can also be displayed by the other components involved in the total matrix. However, their bioactive effect is not of primary importance for the matrix according to the invention.

In the present context a matrix has proven to be particularly suitable whose bioactive component contains 10 to 40% by weight and particularly preferably 15 to 30% by weight phosphatidyl serine. A matrix is also particularly suitable whose bioactive component contains 2.0 to 20% by weight phosphatidyl choline.

Among the relatively broad spectrum of possible compositions, a composition has proven to be particularly advantageous for the claimed matrix which is composed of 10 to 70% by weight and particularly preferably 20 to 50% by weight of the fat component and/or 3 to 30% by weight and particularly preferably 5 to 20% by weight of the wax component, 1 to 30% by weight and particularly preferably 1 to 20% by weight of the polyalcohol component and/or 1.0 to 5% by weight of other physiologically compatible additives.

In addition to the bioactive components phosphatidyl serine and phosphatidyl choline that are essential according to the invention, the matrix can contain as the fat component preferably refined, hydrogenated and/or fractionated fats and in particular those that are rich in omega-3 and/or omega-6 fatty acids such as docosahexaenoic acid, arachidonic acid, eicosapentaenoic acid and conjugated linolenic acid, free fatty acids, in particular omega-3 and omega-6 fatty acids, as the wax component preferably bee wax, candellila wax, shellac, paraffin, monoglycerides or diglycerides and as the polyalcohol component preferably polyethylene glycol, polysorbate, polyglycerol esters, sugar esters or sorbitan esters. The matrix can also advantageously contain tocopherols and derivatives thereof, tocotrienols and derivatives thereof, polycosanols and derivatives thereof, vitamins such as vitamin C and E also in a derivatized form, amino acids in particular the essential, branched and non-proteinogenic amino acids such as theanine, amino acid derivatives such as creatine, taurine, carnitine, phytosterols and derivatives thereof, (poly)phenolic compounds and derivatives thereof such as catechol, phenolic acids such as gallic acid, hydroxycinnamic acids, coumarins, (iso)flavonoids such as quercetin or genistein, lignans and lignins as well as tannin, saponins, mono-, sesqui- and di-terpenes, carotinoids such as beta-carotin, lutein or lycopin, glucosinolates, roughage such as non-starch polysaccharides, extracts of vegetable and/or animal origin, physiologically active proteins such as lactoferrin and glycomacropeptide, phospholipids and glycolipids such as sphingosine or (phyto)sphingomyelin and/or mineral components but also other suitable bioactive components which is also taken into consideration by the present invention.

Phosphatidyl serine and phosphatidyl choline are in particular stabilized according to the invention by other matrix components which are selected such that the total matrix (i.e. composed of PS/PC and the other components) is solid at room temperature and namely to such an extent that when using fats (triglycerides) the solid proportion of the triglyceride that can be determined by TLC is >80% at 23° C. In addition the components are advantageously selected such that the total matrix exhibits the property of shear dilution which for example can be achieved by the preferred use of a combination of fat and wax (e.g. bee wax) in conjunction with PC/PS in the matrix when the triglyceride contains a sufficiently high proportion of solid i.e. unmelted triglycerides. In this case preferred matrix components have a proportion of saturated fatty acids of more than 50% and advantageously no more than four mainly occurring triglyceride species are present. In this connection the use of palm kernel oil together with bee wax has proven to be particularly advantageous.

Hence a person skilled in the art can readily select matrices that are suitable according to the invention on the basis of the criteria 1) the solid proportion of the triglyceride that can be determined by TLC is >80% at 23° C. and 2) the proportion of saturated fatty acids is more than 50%.

In another embodiment of the invention the phosphatidyl serine and phosphatidyl choline are stabilized by using a matrix which contains a polyalcohol component. This matrix can be overall somewhat more liquid where in particular glycerol is added as a polyalcohol component. Disadvantages of conventional lecithin matrices can be overcome in this embodiment. In lecithin matrices the water content is always low due to the strong lipophilic properties of the matrix and the water that is present does not move freely in the matrix but is bound to the polar head groups and hydrates them and/or hydrolyses the head group. This results in the instability of the phospholipid (i.e. PC and/or PS) in conventional matrices. By the addition according to the invention of a polyalcohol component, for example glycerol as a polar substance the water from the head group is displaced and thus prevents or at least delays the hydrolysis of the phospholipid.

Hence in a preferred manner according to the invention the matrix, in addition to PS and PC, contains further at least one fat component and even more preferably a fat component and a wax component and in another embodiment preferably at least one polyalcohol component.

According to the invention particularly suitable matrix materials are substances which enable a complete encapsulation to develop as well as substances which provide a matrix of high stability and low shear stress.

In a preferred embodiment the claimed matrix has a water-containing coat which can also be permanently flexible.

In this context the coat which is preferably also composed of gelatin, glycerol, sugar(alcohols), starch, polysaccharides and mixtures thereof should have a water content of 1.0 to 10.0% by weight based on the total coat. Sorbitol is particularly preferred as a sugar alcohol for the coat and carragenans, alginates and/or pectins are particularly preferred as polysaccharide components. Finally the invention also provides that the coat of the matrix contains silicon dioxide, calcium carbonate, dyes that are suitable for foods, colour pigments and/or talcum as further additives.

Depending on the composition of the bioactive component and the class of substances that make up the coating material, the weight ratio of coat and the bioactive component can be important for the product quality of the total matrix according to the invention. In this context weight ratios of the coat to bioactive component have proven to be particular suitable which are between 1:0.25 to 10.0 and particularly preferably 1:1 to 5.0.

The total diameter of the matrix which should be in particular between 0.3 and 20 mm according to the present invention also depends on the respective intended use.

In addition to the matrix itself the present invention also encompasses its use whereby on the basis of the component contained in the matrix especially strength being the ability to cope with mental or/and physical stress and functional capacity, the prevention of elevated levels of serum cholesterol, the promotion or/and preservation of health and quite generally the improvement of well-being are of primary importance.

Hence with the matrix according to the invention a formulation of phosphatidyl serine and phosphatidyl choline has been found which can be manufactured in an economically acceptable manner and which, in contrast to the other known formulations, has a pronounced oxidative and hydrolytic stability. Thus the matrix according to the invention combines two features which were previously not compatible, namely the ability to be encapsulated and at the same time to have an adequate stability. This combination is especially pronounced in the case of matrices with thixotropic or shear diluting properties. However, it also occurs in matrix forms which are pasty and behave in a Newtonian manner.

The following examples elucidate these advantages of the matrix with a bioactive component containing phospholipid according to the invention.

### EXAMPLE

### Example 1

The hard matrix contained 20% by weight phosphatidyl serine and 15% by weight phosphatidyl choline as the bioactive component. Other components were each 3% by weight phosphatidyl inositol and 2% by weight phosphatidyl ethanolamine, 38% by weight of a mixture of refined soya, two partially hydrogenated soya oils of different melting points as well as 3% by weight of a bee wax, 2% by weight of a mixture composed of vitamins E and D, tocotrieols and β-carotin. The remainder making up 100% by weight was composed of the typical substances accompanying lecithins such as glycolipids, phytosterols and oligosugars.

The two bioactive components were homogenously dispersed in the matrix which was present as pellets having a diameter of 3 to 8 mm.

### Example 2

The hard matrix contained 20% by weight phosphatidyl serine and 5% by weight phosphatidyl choline and 4% by weight phosphatidyl inositol as the bioactive component. Other components were 6% by weight phosphatidyl ethanolamine and 2% by weight phosphatidic acid, 45% by weight palm kernel oil and 5% by weight of a bee wax as well as 0.2% by weight vitamin E. The remainder making up 100% by weight was composed of the typical substances accompanying lecithins such as glycolipids, phytosterols and oligosugars.

### Example 3

The hard matrix contained 30% by weight phosphatidyl serine, 4% by weight phosphatidyl choline and 2% by weight phosphatidyl inositol as the bioactive component. Other components were each 4% by weight phosphatidyl ethanolamine and 1% by weight phosphatidic acid, 45% by weight palm kernel oil and 5% by weight of a bee wax as well as 0.2% by weight vitamin E. The remainder making up 100% by weight was composed of the typical substances accompanying lecithins such as glycolipids, phytosterols and oligosugars.

Stability of the Phospholipids:

Table 1 shows that in the case of phosphatidyl serine (PS) that is very sensitive to hydrolysis and was encapsulated in a soft gelatin capsule as an example, embedding the phospholipids in the matrix according to the invention has a stabilizing effect towards hydrolysis among others.

Two phospholipid-containing lecithins in a standard preparation corresponding to the prior art served as a comparison which were optimized with regard to their ability to be encapsulated by the standard rotary die process. The lecithins were stored in an encapsulated form at room temperature (23° C.).

Examples 1 to 3 are the three described inventive examples; examples 4 to 6 are comparative examples.

TABLE 1

| Ex- | initial value | after 3 months | after 6 months | after 12 months |
|---|---|---|---|---|
| amples | PS [%] | [%] | [%] | [%] |
| 1 | 100 | 99 | 98 | 97 |
| 2 | 100 | 99 | 97 | 96 |
| 3 | 100 | 99 | 98 | 96 |
| 4 | 100 | 84 | 80 | 69 |
| 5 | 100 | 93 | 79 | 70 |
| 6 | 100 | 75 | 64 | 62 |

1. A matrix with a bioactive component containing phospholipid, the matrix containing 5 to 98% by weight phosphatidyl serine (PS) and 1 to 90% by weight phosphatidyl choline (PC) as the bioactive component and in addition 1 to 94% by weight of at least one other component from the series comprising fat component of vegetable and/or animal origin, wax component, polyalcohol component and other physiologically compatible additives, the other matrix components being selected such that the total matrix is solid or paste-like at room temperature and exhibits property of shear dilution.

2. The matrix as claimed in claim 1, wherein the bioactive component contains 10 to 40% by weight of phosphatidyl serine.

3. The matrix as claimed in claim 1, wherein the bioactive component contains 2.0 to 20% by weight of phosphatidyl choline.

4. The matrix as claimed in claim 1, containing 10 to 70% by weight of the fat component and/or 3 to 30% by weight of the wax component and/or 1 to 30% by weight of polyalcohol component and/or 1 to 5% by weight of other physiologically compatible additives.

5. The matrix as claimed in claim 1, wherein it contains refined, hydrogenated and/or fractionated fats and in particular those that are rich in omega-3 and/or omega-6 fatty acids such as docosahexaenoic acid, arachidonic acid, eicosapentaenoic acid and conjugated linolenic acid, free fatty acids, in particular omega-3 and omega-6 fatty acids, bee wax, candellila wax, shellac, paraffin, monoglycerides or diglycerides, polyethylene glycol, polysorbate, polyglycerol esters, sugar esters or sorbitan esters, tocopherols and derivatives thereof, tocotrienols and derivatives thereof, polycosanols and derivatives thereof, vitamins such as vitamin C and E also in a derivatized form, amino acids in particular the essential, branched and non-proteinogenic amino acids such as theanine, amino acid derivatives such as creatine, taurine, carnitine, phytosterols and derivatives thereof, (poly)phenolic compounds and derivatives thereof such as catechol, phenolic acids such as gallic acid, hydroxycinnamic acids, coumarins, (iso)flavonoids such as quercetin or genistein, lignans and lignins as well as tannin, saponins, mono-, sesqui- and di-terpenes, carotinoids such as beta-carotin, lutein or lycopin, glucosinolates, roughage such as non-starch polysaccharides, extracts of vegetable and/or animal origin, physiologically active proteins such as lactoferrin and glycomacropeptide, glycolipids such as sphingosine or (phyto)sphingomyelin and/or mineral components.

6. The matrix as claimed in claim 1, wherein the matrix has a water-containing coat.

7. The matrix as claimed in claim 6, wherein the coat has a water content of 1.0 to 10.0% by weight based on total coat.

8. The matrix as claimed in claim 6, wherein the coat is composed of gelatin, glycerol, sugar (alcohols), starch, polysaccharides and mixtures thereof.

9. The matrix as claimed in claim 8, wherein the coat contains sorbitol as sugar alcohol and carrageenans, alginates and/or pectins as polysaccharide.

10. The matrix as claimed in claim 8, wherein the coat contains silicon dioxide, calcium carbonate, dyes that are suitable for foods, colour pigments and/or talcum as further additives.

11. The matrix as claimed in claim 6, wherein the weight ratio of the coat to bioactive component is 1:0.25 to 10.0.

12. The matrix as claimed in claim 1, having a total diameter of 0.3 to 20 mm.

13. (canceled)

14. (canceled)

15. Method for strengthening ability to cop with mental and/or physical stress and functional capacity, for improving well-being, for promoting and/or preserving health and for preventing elevated levels of serum cholesterol in a subject, comprising steps of:
a) providing a matrix as claimed in claim 1;
b) producing a pharmaceutical preparation comprising said matrix; and
c) administering an effective amount of said pharmaceutical preparation to said subject.

16. The matrix as claimed in claim 1, wherein the bioactive component contains 15 to 30% by weight of phosphatidyl serine.

17. The matrix as claimed in claim 1, containing 20 to 50% by weight of the fat component and/or 5 to 20% by weight of the wax component and/or 2 to 20 % by weight of polyalcohol component and/or 1 to 5% by weight of other physiologically compatible additives.

18. The matrix as claimed in claim 6, wherein the weight ratio of the coat to bioactive component is 1:1 to 5.0.
